(19) United States
(12) Patent Application Publication
(10) Pub. No.: US 2005/0271727 A1
(43) Pub. Date: Dec. 8, 2005

(54) Biodegradable and biocompatible crosslinked polymer hydrogel prepared from PVA and/or PEG macromer mixtures

(75) Inventor: Fei Yao, North Andover, MA (US)

Correspondence Address: Joseph C. Zucchero;Keown & Associates, Suite 1200, 500 West Cummings Park, Woburn, MA, 01801, US

(73) Assignee: Callisyn Pharmaceuticals, Inc.

(21) Appl. No.: 11/145,758

(22) Filed: Jun. 6, 2005

Related U.S. Application Data
(60) Provisional application No. 60/521,615, filed on Jun. 7, 2004.

(51) Int. Cl. A61K 9/14

(52) U.S. Cl. 424/486

(57) ABSTRACT

Biodegradable and biocompatible polymeric hydrogels based on the mixtures of poly(vinyl alcohol) and poly(ethylene glycol) macromers, and methods for their preparation and use, are disclosed. The polymerization may be carried out in situ on organs or tissues or outside the body. Applications for such biocompatible crosslinked hydrogels include prevention of post-operative adhesions, surgical sealants, embolic therapies, controlled delivery of drugs, coating of medical devices such as vascular grafts, wound dressings and other medical applications.

### RELATED APPLICATIONS

This Application claims the benefit of U.S. Provisional Application Ser. No. 60/521,615, filed Jun. 7, 2004. The entire teachings of the above-referenced Application is incorporated herein by reference.

### FIELD OF THE INVENTION

The present invention relates generally to biodegradable and biocompatible hydrogels, and more specifically to biodegradable poly(vinyl alcohol) (PVA) and poly(ethylene glycol) (PEG) mixed hydrogels that are suitable for use as biomaterials.

### BACKGROUND OF THE INVENTION

Biomedical applications of biodegradable and biocompatible hydrogels have become a favored medical practice and have been widely studied. Biodegradable polymers have been used for many years in medical applications. These include sutures, surgical clips, staples, implants, drug delivery systems and others. The majority of these biodegradable polymers have been thermoplastic materials based upon glycolide, lactide, epsilon-caprolactone, and copolymers thereof. Typical examples are the polyglycolide sutures described in U.S. Pat. No. 3,297,033 to Schmitt, the poly(L-lactide-co-glycolide) sutures described in U.S. Pat. No. 3,636,956 to Schneider, the poly(L-lactide-co-glycolide) surgical clips and staples described in U.S. Pat. No. 4,523,591 to Kaplan et al., and the drug-delivery systems described in U.S. Pat. No. 3,773,919 to Boswell et al., U.S. Pat. No. 3,887,699 to Yolles, U.S. Pat. No. 4,155,992 to Schmitt, U.S. Pat. No. 4,379,138 to Pitt et al., and U.S. Pat. Nos. 4,130,639 and 4,186,189 to Shalaby et al.

Traditionally, drugs are administered via oral or injection of high concentrations to achieve therapeutic effects, which often results with adverse side effects. There is urgency to find ways to reduce the need for the systematic administration of high concentration drugs locally. The Biodegradable hydrogels can be carriers for local drug delivery system for biologically active materials such as hormones, enzymes, antibiotics, antineoplastic agents, cell suspensions and other drugs. Therefore, temporary preservation of functional properties of a carried species, as well as controlled release of the species into local tissues or systemic circulation, is possible.

Historically, tissue surgical sealants are used to decrease or prevent the migration of fluid from or into a tissue. A well known material that has been used as a tissue sealant is “fibrin glue”. Fibrin glues have been used extensively in Europe as sealants and adhesives in surgery (Thompson et al., 1988, “Fibrin Glue: A review of its preparation, efficacy, and adverse effects as a topical hemostat,” Drug Intell. and Clin. Pharm., 22:946; Gibble et al., 1990, (1990), “Fibrin glue: the perfect operative sealant?” Transfusion, 30(8):741). Fibrin glue is typically made by contacting a solution or suspension of the blood protein fibrinogen with an enzyme or other reagent which will cause fibrin to crosslink. However, Fibrin glues have not been used extensively in the United States due to concerns relating to disease transmission from blood products, such as AIDS, Hepatitis, Mad Cow diseases, etc. An obvious disadvantage of this product is that it may cause an immune reaction in patients who are sensitive to collagen or gelatin.

Post-surgical adhesions formation involves organs of the peritoneal cavity. The peritoneal wall is a frequent and undesirable result of abdominal surgery. Surgical trauma to the tissue caused by handling and drying results in release of a serosanguinous (proteinaceous) exudate which tends to collect in the pelvic cavity (Holtz, G., 1984). If the exudate is not absorbed or lysed within this period it becomes ingrown with fibroblasts, and subsequent collagen deposition leads to adhesion formation.

Approaches to eliminate adhesion formation have been attempted, with limited success in most cases. These approaches included lavage of the peritoneal cavity, administration of pharmacological agents, and the application of barriers to mechanically separate tissues. For example, Poloxamer 407 has been used for the treatment of adhesions, with some success. Poloxamer is a copolymer of ethylene oxide and propylene oxide and is soluble in water; the solutions are liquids at room temperature. Oxidized regenerated cellulose have been used to prevent adhesions and is an approved clinical product, trade-named INTERCEED TC7. It was shown to be more effective if pretreated with heparin, but was still unable to completely eliminate adhesions (Diamond et al., “Synergistic effects of INTERCEED (TC7) and heparin in reducing adhesion formation in the rabbit uterine born model,” Fertility and Sterility 55(2):389 (1991)).

Biomaterials occluding blood vessels, occluding other body lumens such as fallopian tubes, filling aneurysm sacs, as arterial sealants, and as puncture sealants are called embolic agents. Blood vessels embolization is performed for a number of reasons. One of them is to reduce blood flow to and encourage atrophy of tumors, such as in the liver. Another is to reduce blood flow and induce atrophy of uterine fibroids, for treatment of vascular malformations, such as arteriovenous malformations (AVMs) and arteriovenous fistulas (AVFs), to seal endoleaks into aneurysm sacs, to stop uncontrolled bleeding, or to slow bleeding prior to surgery.

Temporary occlusion is desirable, for example, in treating of tumors, to allow for recanalization and reapplication of a chemotherapeutic agent to the tumor. As another example, temporary occlusion may be desirable when using the embolic composition for temporary sterilization. Temporary occlusion can be achieved by using a fully degradable embolic composition or a composition degraded in response to an applied condition, such as a change in temperature or pH.

Many surgical procedures are now performed in a minimally invasive fashion that reduces morbidity associated with the procedure. Minimally invasive surgery encompasses laparoscopic, thoracoscopic, arthroscopic, intraluminal endoscopic, endovascular, interventional radiological, catheter-based cardiac (such as balloon angioplasty), and like techniques. These procedures allow mechanical access to the interior of the body with the least possible perturbation of the patient's body.

Most of the polymers used with minimally invasive surgery applications are pre-formed to a specific shape before being used in a given application. However, such pre-formed objects have limitations in minimally invasive surgery because they, like other large objects, are difficult to transport through the small access sites afforded by minimally invasive surgery techniques. In addition, the shape of the pre-formed object may not be appropriate because the target tissues where such objects are likely to be used have a variety of shapes and sizes.

To overcome these limitations, in situ curable or gelable biocompatible crosslinked polymer systems have been explored. The precursors of such systems are usually liquid in nature. These liquids are then transported to the target tissue and applied on the target organ or tissue. The liquid flows and conforms to the shape of the target organ. The shape of the conformed liquid is then preserved by polymerization or a gelation reaction. This approach has several advantages, including conformity to organ shapes and the ability to implant large quantities of liquid using minimally invasive surgery procedures.

U.S. Pat. No. 5,410,016 to Hubbell et al. discloses biocompatible, biodegradable macromers which can be polymerized to form hydrogels. The macromers are block copolymers that include a biodegradable block, a water-soluble block with sufficient hydrophilic character to make the macromer water-soluble, and one or more polymerizable groups. The polymerizable groups are separated from each other by at least one degradable group. One of the disclosed uses for the macromers is to plug or seal leaks in tissue.

Other hydrogels have been described, for example, in U.S. Pat. No. 4,938,763 to Dunn et al., U.S. Pat. Nos. 5,100,992 and 4,826,945 to Cohn et al., U.S. Pat. Nos. 4,741,872 and 5,160,745 to De Luca et al., U.S. Pat. No. 5,527,864 to Suggs et al., and U.S. Pat. No. 4,511,478 to Nowinski et al. Methods of using such polymers are described in U.S. Pat. No. 5,573,934 to Hubbell et al. and PCT WO 96/29370 by Focal.

The major disadvantage of the macromers and hydrogels disclosed by Hubbell is that they are inflexible in design. PEG has only two groups which are easily modified, the terminal hydroxyl groups, and those groups are modified with the biodegradable and polymerizable groups. Also, the degradable PEG material developed by Hubbell et al. exhibits a large degree of swelling in aqueous solutions, which is disadvantageous in many applications.

PVA based hydrogels are disclosed in U.S. Pat. Nos. 5,508,317 and 5,932,674 to Muller. However, these hydrogels are not degradable. U.S. Pat. No. 6,710,126 to Hirt et al. discloses hydrogel made of prepolymers having a PVA backbone and pendant chains that include a polymerizable group. In one embodiment, the pendant chains also include a biodegradable region. In another embodiment, biodegradable regions are incorporated into the hydrogel during its formation.

As Hirt et al. stated, PVA hydrogels offer many advantages over PEG based hydrogels. For example, the availability of pendant OH groups along a PVA backbone adds versatility in terms of the various modifications that could be made to the macromer (e.g. attachment of degradable segments, active agents, hydrophobic groups, etc). With a PVA hydrogel, the choice of a suitable PVA (with appropriate attached groups if desired) can yield a non-swellable, minimally swellable, or even shrinkable system. PVA possesses greater adhesive properties than PEG. This might be desirable for certain applications. Furthermore, PVA due to its hydrocarbon backbone has greater oxidative stability than PEG and it can be stored as aqueous solutions as opposed to PEG that has to be stored as a freeze-dried powder.

A disadvantage of the PVA hydrogels that have been developed is that their mechanical properties are not desirable, such as low elasticity, high modulus, and more brittle, comparing with PEG based hydrogels.

Accordingly, it would be advantageous to have a hydrogel with both PVA and PEG hydrogels' advantages and properties such as biodegradable, biocompatible, easily modifiable, multi-functionable, elastic and durable, minimally swellable, and greater adhesive. Moreover, it would be advantageous to have a degradable hydrogel having multiple pendant groups that allow for the attachment of various modifiers.

### SUMMARY OF THE INVENTION

It is therefore an object of this invention to provide such crosslinked polymer hydrogels preferably is biodegradable and biocompatible, and can be designed with selected properties of compliancy (i.e., high elastic modulus and low elongation at rupture) and elasticity for different surgical and therapeutic applications.

It is another object of the present invention to provide biocompatible crosslinked polymer hydrogels and methods for their preparation and use, in which the biocompatible crosslinked polymer hydrogels are formed using both PVA and/or PEG macromers.

It is also an object of this invention to provide such biocompatible crosslinked polymer hydrogels and methods for their preparation and use, in which the biocompatible crosslinked polymer hydrogels are formed from aqueous solutions, preferably under physiological conditions.

It is still another object of this invention to provide such biocompatible crosslinked polymer hydrogels and methods for their preparation and use, in which the biocompatible crosslinked polymer hydrogels are formed in vivo and in vitro.

It is still a further object of this invention, to provide such biocompatible crosslinked polymer hydrogels and methods for their preparation and use, in which the biocompatible crosslinked polymer hydrogels are biodegradable.

It is yet another object of this invention to provide methods for preparing tissue conforming, biocompatible crosslinked polymer hydrogels in a desirable form, size and shape.

It is yet a further object of this invention to provide methods for using biocompatible crosslinked polymer hydrogels to form medically useful devices or implants for use as surgical adhesion prevention barriers, as implantable wound dressings, as scaffolds for cellular growth for tissue engineering, or as surgical tissue adhesives or sealants and other medical applications.

Another object of this invention is to provide methods for using biocompatible crosslinked polymers to form medically useful devices or implants that can release bioactive compounds in a controlled manner for local, systemic, or targeted drug delivery.

### DETAILED DESCRIPTION OF THE PREFERRED EMBODIMENTS

Biodegradable hydrogels based on poly(vinyl alcohol) (PVA) and/or poly(ethylene glycol) (PEG) have been developed which can be rapidly formed in an aqueous surrounding, e.g., in vivo. The PVA and PEG based hydrogels can be designed to degrade as fast as a few days to more than 1 year. Degradation rates are determined in one respect by selection of an appropriate degradable region. Other factors that will affect the degradation rate are the density of the pendant chain bearing the degradable region, the length of the degradable region, the hydrophobicity of the network, the mixing ratio of PVA/PEG polymers, and the crosslinking density.

The PVA and PEG based hydrogels can be designed to be flexible in mechanical properties such as elasticity, durability, and adhesiveness. Mechanical properties are determined by the molecular weight, mixing ratio of PVA/PEG polymers, the density of the pendant chain bearing the degradable region, the hydrophobicity of the network, and the crosslinking density.

The PVA and PEG based hydrogels can be prepared in many different ways. For example, they can be formed by crosslinking two components. Component A is a PVA backbone having pendant chains with crosslinkable groups, and component B is a PEG having a degradable region flanked by crosslinkable groups.

The PVA and PEG based hydrogels can also be formed by crosslinking two components. Component A is a PEG backbone with crosslinkable groups, and component B is a PVA backbone having pendant chains with a degradable region flanked by crosslinkable groups.

The PVA and PEG based hydrogels can also be formed by crosslinking two components. Component A is a PEG backbone and component B is a PVA backbone, both having pendant chains with a degradable region flanked by crosslinkable groups.

The PVA and PEG based hydrogels can also be formed by crosslinking two components. Component A is either a PEG or a PVA backbone with crosslinkable groups, and component B is crosslinkers with biodegradable regions end caped with crosslinking groups.

Functional Groups

At least one component is multifunctional, meaning that it comprises at least one or more biodegradable and crosslinkable functional groups, such that a component with crosslinkable functional group may react with another component with at least one biodegradable and crosslinkable functional groups to form a covalent bond which is biodegradable. Such reactions are referred to as “crosslinking reactions”. Preferably, each component comprises both biodegradable and crosslinkable groups, so long as both components are used in the crosslinking reaction.

The PVA and PEG based hydrogels can be polymerized via any of a number of means, such as physical crosslinking or chemical crosslinking. Chain reaction polymerization includes but is not exclusive to free radical polymerization (thermal, photo, redox, atom transfer polymerization, etc.), cationic polymerization (including onium), anionic polymerization (including group transfer polymerization), certain types of coordination polymerization, certain types of ring opening and metathesis polymerizations, etc.

Photoinitiated polymerization initiator system: Useful photoinitiators are those which can be used to initiate by free radical generation polymerization of the macromers without cytotoxicity and within a short time frame, minutes at most and most preferably seconds. Preferred initiators of choice for UV or Visible light initiation are irgacures, ethyl eosin, 2,2-dimethoxy-2-phenyl acetophenone, other acetophenone derivatives, and camphorquinone. In all cases, crosslinking and polymerization are initiated among macromers by a light-activated free-radical polymerization initiator such as 2,2-dimethoxy-2-phenylacetophenone or a combination of ethyl eosin and triethanol amine, for example.

Thermal polymerization initiator systems: Such systems that are unstable at 37 degree C. and would initiate free radical polymerization at physiological temperatures include, for example, potassium persulfate, with or without tetraamethyl ethylenediamine; benzoylperoxide, with or without triethanolamine; and ammonium persulfate with sodium bisulfite. Other peroxygen compounds include t-butyl peroxide, hydrogen peroxide and cumene peroxide.

Redox polymerization initiators system: Metal ions can be either an oxidizer or a reductant in systems including redox initiators. For example, ferrous ion is used in combination with a peroxide to initiate polymerization, or as parts of a polymerization system. In this case the ferrous ion is serving as reductant. Other systems are known in which a metal ion acts as oxidant. For example, the ceric ion (4+valence state of cerium) can interact with various organic groups, including carboxylic acids and urethanes, to remove an electron to the metal ion, and leaving an initiating radical behind on the organic group. Here the metal ion acts as an oxidizer. Potentially suitable metal ions for either role are any of the transition metal ions, lanthanides and actinides, which have at least two readily accessible oxidation states. Preferred metal ions have at least two states separated by only one difference in charge. Of these, the most commonly used are ferric/ferrous; cupric/cuprous; ceric/cerous; cobaltic/cobaltous; vanadate V vs. IV; permanganate; and manganic/manganous.

Definitions

A “hydrogel” is a substance formed when an organic polymer (natural or synthetic) is cross-linked via covalent, ionic, or hydrogen bonds to create a three-dimensional open-lattice structure which entraps water molecules to form a gel.

A “sealant” is a material which decreases or prevents the migration of fluid from or into a tissue. Sealants are typically applied to a tissue and then locally crosslinked or otherwise processed. The same materials may also be used to adhere structures or tissues together, either when applied between them and crosslinked or processed, or when used to encase junctions of tissue and/or devices.

“Crosslink” is used generically to refer to the joining of smaller entities to form a structure by any physical or chemical means, such as a reactive functional group that has the capacity to form additional covalent bonds resulting in macromer interlinking. Polymerizable groups specifically include groups capable of polymerizing via free radical polymerization and groups capable of polymerizing via cationic or heterolytic polymerization. Unless stated otherwise, the terms “polymerize” and “gel” are functional equivalents of “crosslink”.

“Biocompatibility”, in the context of the materials and devices of the invention, is the absence of stimulation of a severe, long-lived or escalating biological response to an implant or coating, and is distinguished from a mild inflammation which typically accompanies surgery or implantation of foreign objects into a living organism.

“Biodegradability”, in the context of the materials and devices of the invention, is the predictable disintegration of an implant into entities which will be metabolized or excreted, under the conditions normally present in a mammalian organism or living tissue.

“Water-soluble” refers to a material soluble to at least 1% by weight in water or an aqueous solution. It is defined herein as a solubility of at least one gram/liter in an aqueous solution at a temperature in the range of about 0.degree. C. and 50.degree. C.

“Aqueous solutions” can include small amounts of water-soluble organic solvents, such as dimethylsulfoxide, dimethylformamide, alcohols, acetone, and/or glymes.

“Macromers” or “Monomers” or “Prepolymers” mentioned herein are polymers that are soluble in aqueous solutions, or nearly aqueous solutions, such as water with added dimethylsulfoxide. They have a water soluble region flanked by zero or more functional groups such as biodegradable region, preferably hydrolyzable under in vivo conditions, and at least one or more polymerizable regions.

“Materials Properties”, are the properties of the biomaterials and hydrogels disclosed herein and include:
“Young's modulus” (of elasticity) which is the limiting modulus of elasticity extrapolated to zero strain;
“Elastic modulus” which is any modulus of elasticity, not limited to Young's modulus, and may include “secant modulus” and other descriptors of non-linear regions of the stress-strain curve;
“Bulk” or “compressive” modulus which is used in its usual sense of: ratio of stress to a designated compressive strain;
“Elongation at failure” which is the relative strain or extension of a test specimen at which any irreversible or hysteresis-inducing change occurs in the specimen; and
“Elongation at break” or “elongation at rupture” which is the relative strain (extension) of a test specimen at which mechanical rupture occurs.

“Compliance” as used herein is used in a general sense, and refers for example to the ability of an implant to closely match the physiological and mechanical properties of tissues at the implant site, except when “compliance” is used in a specific technical sense as the reciprocal of a modulus.

Component A: Prepolymer PVA or PEG Backbone With Polymerizable Groups

In the embodiments, the core water soluble region can consist of poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propyleneoxide) block copolymers (poloxamers and meroxapols), poloxamines, carboxymethyl cellulose, hydroxyalkylated celluloses such as hydroxyethyl cellulose and methylhydroxypropyl cellulose, polysaccharides or carbohydrates such as hyaluronic acid, Ficoll® polysucrose, dextran, heparan sulfate, chondroitin sulfate, heparin, or alginate, proteins such as polypeptides, polynucleotides, gelatin, collagen, albumin, ovalbumin, or polyamino acids. Properties of the monomer, other than polymerizability, will be selected according to the use, using principles as known in the art. Preferably, the water-soluble polymeric blocks are made from poly(ethylene glycol) or poly(ethylene oxide) and poly(vinyl alcohol).

PVA

Polyvinyl alcohols which can be used as prepolymer backbones are commercially available PVAs, for example Vinol.RTM. 107 from Air Products (MW=22,000 to 31,000 Da, 98-98.8% hydrolyzed), Polysciences 4397 (MW=25,000 Da, 98.5% hydrolyzed), BF 14 from Chan Chun, Elvanol.RTM. 90-50 from DuPont and UF-120 from Unitika. Other producers are, for example, Nippon Gohsei (Gohsenol.RTM.), Monsanto (Gelvatol.RTM.), Wacker (Polyviol.RTM.) or the Japanese producers Kuraray, Deriki, and Shin-Etsu. In some cases it is advantageous to use Mowiol.RTM. products from Hoechst, in particular those of the 3-83, 4-88, 4-98, 6-88, 6-98, 8-88, 8-98, 10-98, 20-98, 26-88, and 40-88 types.

It is also possible to use copolymers of hydrolyzed or partially hydrolyzed vinyl acetate, which are obtainable, for example, as hydrolyzed ethylene-vinyl acetate (EVA), or vinyl chloride-vinyl acetate, N-vinylpyrrolidone-vinyl acetate, and maleic anhydride-vinyl acetate. If the prepolymer backbones are, for example, copolymers of vinyl acetate and vinylpyrrolidone, it is again possible to use commercially available copolymers, for example the commercial products available under the name Luviskol.RTM. from BASF. Particular examples are Luviskol VA 37 HM, Luviskol VA 37 E and Luviskol VA 28. If the prepolymer backbones are polyvinyl acetates, Mowilith 30 from Hoechst is particularly suitable.

The starting polyvinyl alcohols preferably have a mean molecular weight of at least 2000 Da. Polyvinyl alcohols that can be derivatized in accordance with the invention preferably have a molecular weight of at least 10,000 Da. As an upper limit, the polyvinyl alcohols may have a molecular weight of up to 1,000,000 Da. Preferably, the polyvinyl alcohols have a molecular weight of up to 300,000 Da, especially up to approximately 100,000 Da and especially preferably up to approximately 30,000 Da.

The polyvinyl alcohols usually have a poly(2-hydroxy)ethylene structure. The polyvinyl alcohols derivatized in accordance with the disclosure may, however, also comprise hydroxy groups in the form of 1,2-glycols.

The PVA system can be a fully hydrolyzed PVA, with all repeating groups being
—CH.sub.2-CH(OH)
or a partially hydrolyzed PVA with varying proportions (25% to 1% ) of pendant ester groups. PVA with pendant ester groups have repeating groups of the structure
—CH.sub.2-CH(OR)
or in a brief form
PVA-(OR)
where PVA is —CH.sub.2CH repeating units, R is H, COCH.sub.3 group or longer alkyls, as long as the water solubility of the PVA is preserved. The ester groups can also be substituted by acetaldehyde or butyraldehyde acetals that impart a certain degree of hydrophobicity and strength to the PVA. For an application that requires an oxidatively stable PVA, the commercially available PVA can be broken down by
NalO.sub.4-KMnO.sub.4
oxidation to yield a small molecular weight (3-4K) PVA.

The PVAs are prepared by basic or acidic, partial or virtually complete hydrolysis of polyvinyl acetate. In a preferred embodiment, the polyvinyl alcohol derivatized in accordance with the invention comprises less than 50% of vinyl acetate units, especially less than about 25% of vinyl acetate units. Preferred amounts of residual acetate units in the polyvinyl alcohol derivatized in accordance with the invention, based on the sum of vinyl alcohol units and acetate units, are approximately from 3 to 25%.

PEG

Covalent attachment of the hydrophilic polymer poly(ethylene glycol), abbreviated PEG, also known as poly(ethylene oxide), abbreviated PEO, to molecules and surfaces is of considerable utility in biotechnology and medicine. In its most common form, PEG is a linear polymer terminated at each end with hydroxyl groups:
HO—CH.sub.2CH.sub.2 O—(CH.sub.2CH.sub.2O).sub.n —CH.sub.2 CH.sub.2 —OH

The above polymer, alpha-,omega-dihydroxylpoly(ethylene glycol), can be represented in brief form as
HO-PEG-OH
where it is understood that the -PEG-symbol represents the following structural unit:
—CH.sub.2CH.sub.2O—(CH.sub.2CH.sub.2O).sub.n —CH.sub.2CH.sub.2-
where n typically ranges from about 3 to about 4000.

PEG is a polymer having the properties of solubility in water and in many organic solvents, and has received the most interest because of its absence of toxicity, antigenicity, immunogenicity, for its degree of amphiphilicity. PEG can be activated at each terminus to be bifunctional. PEG can also be modified to have a reactive moiety at one end such as commonly used methoxy-PEG-OH, or mPEG in brief, in which one terminus is the relatively inert methoxy group, while the other terminus is a hydroxyl group that is subject to ready chemical modification.

It should be understood that the use of the term PEG or poly(ethylene glycol) is intended to be inclusive and not exclusive in this respect. The term PEG includes poly(ethylene glycol) in any of its forms, including alkoxy PEG, difunctional PEG, multiarmed PEG, forked PEG, branched PEG, pendent PEG (i.e. PEG or related polymers having one or more functional groups pendent to the polymer backbone), or PEG with degradable linkages therein. At all except the lowest molecular weights, poly(ethylene glycol) has a broad molecular weight distribution ranging from ˜0.5× to 1.5×.

PEG which can be used as prepolymer backbones are commercially available PEGs or PEOs, for example, PEGs and PEOs from Union Carbide, Fluka ,Polysciences and Crescent Chemical, Carbowax products from Dow, US19959-1 kg from Amersham Biosciences (MW=8,000 Da), and products from other suppliers.

Polyethylene glycols that can be used in accordance with the invention preferably have a molecular weight of at least 100 Da. As an upper limit, the polyethylene glycols may have a molecular weight of up to 1,000,000 Da. Preferably, the polyethylene glycols have a molecular weight of up to 300,000 Da, especially up to approximately 100,000 Da and especially preferably up to approximately 50,000 Da.

Crosslinkable Groups

The crosslinkable end groups contain a carbon-carbon double bond capable of polymerizing the macromers as illustrated the simplest form below.
—CO—CH═CH.sub.2

The macromers have at least two pendant chains containing groups that can be crosslinked. The term group includes single polymerizable moieties, such as an acrylate, as well as larger crosslinkable regions, such as oligomeric or polymeric regions. The macromers can contain more than one type of crosslinkable group. The pendant chains are attached via the hydroxyl groups of the polymer backbone.

The crosslinkable groups consist preferably of the following groups: acrylates, diacrylates, oligoacrylates, (meth)acrylamide, (meth)acrylates, dimethacrylates, oligomethoacrylates, styryl, vinyl ester, vinyl ketone, vinyl ethers, or other biologically acceptable polymerizable groups.

Simply, the prepolymers A stated above can be represented in brief form as
PEG-crosslinkable groups
   or
   PVA-(OR)
   where R is H, COCH.sub.3 group or longer alkyls, and/or crosslinkable groups.

One simple example is acrylate-capped polyethylene glycol (PEG-diacrylates). The other examples are polymers containing ethylenically-unsaturated groups, such as those of U.S. Pat. No 4,938,763 to Dunn et al., U.S. Pat. Nos. 5,100,992 and 4,826,945 to Cohn et al., U.S. Pat. Nos. 4,741,872 and 5,160,745 to De Luca et al., U.S. Pat. No. 5,410,016 to Hubbell et al., U.S. Pat. No. 5,932,674 to Muller, U.S. Pat. No. 6,566,406 to Pathak et al., and U.S. Pat. No. 6,710,126 to Hirt et al.

The prepolymers can be made by general synthetic methods known to those skilled in the art. Some prepolymers can be purchased commercially in the market such as PEG-diacrylates.

Component B: Prepolymer PVA or PEG Backbone with Crosslinkers Including Degradable Region Flanked by Polymerizable Groups

Component B is a molecule having crosslinkers which including a degradable region flanked by crosslinkable groups. Component B contains at least 1 group capable of crosslinking with the crosslinkable groups of component A. If Component B is based on PVA, Component B also includes at least one group capable of crosslinking with other components B after they have been attached to component A. The mechanism by which component A crosslinks with component B can be different than the mechanism by which component B crosslinks with other component B's after they are attached to component A's. When component A is a specific prepolymer as described above, component B includes a group capable of crosslinking with the olefinically unsaturated group of component A. Component B can include other copolymers in addition to the degradable region and crosslinkable group.

Crosslinkable Groups

Any of the crosslinkable groups described above with respect to component A can also be used on component B. Different types of crosslinking may be employed for crosslinking of A to one end of B and of B with other B's.

Degradable Groups

The degradable region is preferably degradable under in vivo conditions by hydrolysis or enzymes. For example, the degradable region may be polymers and oligomers of glycolide, lactide, epsilon-caprolactone, other hydroxy acids, and other biologically degradable polymers that yield materials that are non-toxic or present as normal metabolites in the body. Preferred poly(alpha-hydroxy acid)s are poly(glycolic acid), poly(DL-lactic acid) and poly(L-lactic acid). Other useful materials include poly(amino acids), poly(anhydrides), poly(orthoesters), poly(orthocarbonates), poly(phosphazines), and poly(phosphoesters). Polylactones such as poly(epsilon-caprolactone), poly(delta-valerolactone), poly(gamma-butyrolactone), and poly(trimethylene carbonate), for example, are also useful. Enzymatically degradable linkages include poly(amino acids), gelatin, chitosan, and carbohydrates. The biodegradable regions may have a degree of polymerization ranging from one up to values that would yield a product that was not substantially water soluble. Thus, monomeric, dimeric, trimeric, oligomeric, and polymeric regions may be used. The biodegradable region could, for example, be a single methacrylate group. The degradable region may include a copolymer of at least two different monomers or a blend of at least two different monomers.

Biodegradable regions can be constructed from polymers or monomers using linkages susceptible to biodegradation, such as ester, acetal, carbonate, peptide, anhydride, orthoester, phosphazine, and phosphoester bonds.

Crosslinkers

The biodegradable groups end caped with crosslinkable groups are called crosslinkers, which itself is biodegradable and crosslinkable. In a preferred embodiment, the biodegradable groups include an end cap on one side. The end cap comprises one or more functional groups capable of cross-linking the macromers. The crosslinkers can be made by general synthetic methods known to those skilled in the art. The crosslinkers can be represented simply as
-R.sub.1 —CO—CH═CH.sub.2
where R.sub.1 is part of the biodegradable region and can be a wide choices for those of skill in the art, as stated in the Biodegradable Groups section. A simple example of crosslinker is acrylated glycine anhydride type compound:
CH.sub.2=CH—CO—NH—CH.sub.2-CO—O—CO—CH.sub.2-NH—CO—CH═CH.sub.2

The PVA and PEG backbones attached with the crosslinkers are biodegradable and polymerizable. Simply, the prepolymers B stated above could be represented in brief form as
PEG-crosslinkers
or
PVA-(OR)
   where R is H, COCH.sub.3 group or longer alkyls, and/or crosslinkers;
   or
crosslinkers

The macromers are crosslinkable in an extremely effective and controlled manner. The macromers can be synthesized using means well known to those of skill in the art. General synthetic methods analogous to those are found in the literature, for example in U.S. Pat. No. 5,410,016 to Hubbell et al., U.S. Pat. No. 4,243,775 to Rosensaft et al., U.S. Pat. No. 4,526,938 to Churchill et al., U.S. Pat. No. 6,083,524 to Sawhney et al., U.S. Pat. No. 6,566,406 to Pathak et al., and U.S. Pat. No. 6,710,126 to Hirt et al.

Those skilled in the art will recognize that oligomers of the core, extensions and crosslinkers may have uniform compositions or may be combinations of relatively short chains or individual species which confer specifically desired properties on the final hydrogel while retaining the specified overall characteristics of each section of the macromer. The lengths of oligomers referred to herein may vary from two mers to many, the term being used to distinguish subsections or components of the macromer from the complete entity.

The specific macromers described above are extraordinarily stable. Spontaneous crosslinking by homopolymerization does not typically occur. The macromers can furthermore be purified in a manner known per se, for example by precipitation with organic solvents, such as acetone, extraction in a suitable solvent, washing, dialysis, filtration, or ultrafiltration. Ultrafiltration is especially preferred. By means of the purification process the macromers can be obtained in extremely pure form, for example in the form of concentrated aqueous solutions that are free, or at least substantially free, from reaction products, such as salts, and from starting materials.

The preferred purification process for the macromers of the invention, ultrafiltration, can be carried out in a manner known per se. It is possible for the ultrafiltration to be carried out repeatedly, for example from two to ten times. Alternatively, the ultrafiltration can be carried out continuously until the selected degree of purity is attained. The selected degree of purity can in principle be as high as desired. A suitable measure for the degree of purity is, for example, the sodium chloride content of the solution, which can be determined simply in a known manner, such as by conductivity measurements.

Methods of Making Biodegradable PVA and PEG Mixed Hydrogels from Components A and B

The methods of making a hydrogel from components A and B involves combining the components under conditions suitable for crosslinking of components A and B and, optionally in a second step, crosslinking of components B after they have been attached to components A. It is preferred that the component A is
PEG-crosslinkable groups
   and component B is
PVA-(OR)
   where R is H, COCH.sub.3 group or longer alkyls, and/or crosslinkers. The following descriptions and examples are based on this preferred combination.

The crosslinking is suitably carried out in a solvent, preferably under physiological conditions. A suitable solvent is in principle any solvent that dissolves components A and B, for example water, alcohols, such as lower alkanols, for example ethanol or methanol, also carboxylic acid amides, such as dimethylformamide, or dimethyl sulfoxide, and also a mixture of suitable solvents, such as, for example, a mixture of water with an alcohol, such as, for example, a water/ethanol or a water/methanol-mixture. The combination of A and B is preferably carried out in a substantially aqueous solution. In accordance with the invention, the criterion that the prepolymer is soluble in water denotes in particular that the prepolymer is soluble in a concentration of approximately from 3 to 80% by weight, preferably approximately from 5 to 70% by weight, in a substantially aqueous solution. Insofar as it is possible in an individual case, prepolymer concentrations of more than 80% are also included in accordance with the invention.

Within the scope of this invention, substantially aqueous solutions of the prepolymer comprise especially solutions of the prepolymer in water, in aqueous salt solutions, especially in aqueous same solutions that have an osmolarity of approximately from 200 to 450 milliosmol per 1000 ml (unit: mOsm/l), preferably an osmolarity of approximately from 250 to 350 mOsm/l, especially approximately 300 mOsm/l, or in mixtures of water or aqueous salt solutions with physiologically tolerable polar organic solvents, such as, for example, glycerol. Solutions of the prepolymer in water or in aqueous salt solutions are preferred.

Components A and B are preferably combined such that a hydrogel is formed having crosslinking in an amount of from approximately 0.25 to 10 milliequivalents of crosslinker per gram of PVA (meq/g), more desirably about 0.25 to 1.5 meq/g.

Components A and B are preferably combined such that a hydrogel is formed having the macromer ratios of PVA/PEG that will generate a suitable hydrogel for the specific applications. The hydrogel will possess the desired properties such as elasticity, durability, minimally swellability, and greater adhesiveness.

In order to encourage inter crosslinking between A and B prior to intra crosslinking of B with B, a large excess of B can be used, such as a ten fold increase. It is possible that a partially degradable hydrogel will result from this system. Such a partially degradable hydrogel may be desirable for some applications.

Preferably, the prepolymers used in the process according to the invention can be purified in a manner known per se, for example by precipitation with organic solvents, such as acetone, filtration and washing, extraction in a suitable solvent, dialysis or ultrafiltration, ultrafiltration being especially preferred. By means of that purification process the prepolymers can be obtained in extremely pure form, for example in the form of concentrated aqueous solutions that are free, or at least substantially free, from reaction products, such as salts, and from starting materials, such as, for example, non-polymeric constituents.

The preferred purification process for the prepolymers used in the process according to the invention, ultrafiltration, can be carried out in a manner known per se. It is possible for the ultrafiltration to be carried out repeatedly, for example from two to ten times. Alternatively, the ultrafiltration can be carried out continuously until the selected degree of purity is attained. The selected degree of purity can in principle be as high as desired. A suitable measure for the degree of purity is, for example, the sodium chloride content of the solution, which can be determined simply in a known manner, such as by conductivity measurements.

One additive that is added, where appropriate, to the solution of the prepolymer is an initiator for the crosslinking, should an initiator be required for crosslinking the crosslinkable groups. Moreover, it may be desirable to employ different crosslinking means for crosslinking component A to component B and for crosslinking component B to other component B's after they are attached to component A's. For example, it may be desirable to employ salt crosslinking for crosslinking component A to component B but to employ redox initiated free radical crosslinking for crosslinking components B.

Characteristics That Can Be Modified

The compositions are highly versatile. A number of characteristics can be easily modified, making the compositions suitable for a number of applications. For example, as discussed above, the polymer backbones can include comonomers to add desired properties, such as, for example, thermoresponsiveness, degradability, gelation speed, and hydrophobicity. Modifiers can be attached to the polymer backbone (or to pendant groups) to add desired properties, such as, for example, thermoresponsiveness, degradability, hydrophobicity, and adhesiveness. Active agents can also be attached to the polymer backbone using the free hydroxyl groups, or can be attached to pendant groups.

The viscosity of the solution of the prepolymer in the substantially aqueous solution is, within wide limits, not critical, but the solution should preferably be a flowable solution that can be deformed strain-free.

The molecular weight of the prepolymer is also, within wide limits, not critical. Preferably, however, the prepolymer A has a molecular weight of from approximately 100 to approximately 500,000 Da, most preferably from about 100 to 30,000 Da. The prepolymer B has a molecular weight of from approximately 2,000 to approximately 500,000 Da, most preferably from about 3,000 to 30,000 Da.

The gelation time of the liquid compositions can be varied from about 0.5 seconds to as long as 10 minutes, and longer if desired. A longer gelation time will generally be required if crosslinking is initiated a distance from the intended application site.

The gelation time will generally be affected by, and can be modified by changing at least the following variables: the initiator system, crosslinker density, macromer molecular weight, macromer concentration (solids content), and type of crosslinker. A higher crosslinker density will provide faster gelation time; a lower molecular weight will provide a slower gelation time. Higher solids content will provide faster gelation time.

For redox systems the gelation time can be designed by varying the concentrations of the redox components. Higher reductant and higher oxidant will provide faster gelation, higher buffer concentration and lower pH will provide faster gelation.

The firmness of the formed hydrogel will be determined in part by the hydrophilic/hydrophobic balance, where a higher hydrophobic percent provides a firmer hydrogel. The firmness will also be determined by the crosslinker density (higher density provides a firmer hydrogel), the macromer molecular weight (lower MW provides a firmer hydrogel), and the length of the crosslinker (a shorter crosslinker provides a firmer hydrogel).

The swelling of the hydrogel is inversely proportional to the crosslinker density. Generally, no or minimal swelling is desired, desirably less than about 10 percent.

Elasticity of the formed hydrogel can be increased by increasing the size of the backbone between crosslinks and decreasing the crosslinker density. Incomplete crosslinking will also provide a more elastic hydrogel. Preferably the elasticity of the hydrogel substantially matches the elasticity of the tissue to which the composition is to administered.

The ratio of PVA macromer vs. PEG macromer will also determine the hydrogel properties mentioned above. Appropriate mixture of the macromers will generate a suitable hydrogel for the specific applications. The hydrogel will possess the desired properties such as elasticity, durability, minimally swellability, and greater adhesiveness.

General Analysis

The prepolymers synthesized were chemically analyzed using structure-determining methods such as nuclear (proton and carbon-13) magnetic resonance spectroscopy, infrared spectroscopy. Molecular weights were determined using high pressure liquid chromatography and gel permeation chromatography. Thermal characterization of the polymers, including melting point and glass transition temperatures, were performed using differential scanning calorimetric analysis. Aqueous solution properties such as micelle and gel formation were determined using fluorescence spectroscopy, UV-visible spectroscopy and laser light scattering instruments.

In vitro degradation of the polymers was followed gravimetrically at 37.degree. C., in an aqueous buffered medium such as phosphate buffered saline (at pH 7.2). In vivo biocompatibility and degradation life times were assessed by injecting or forming a gelling formulation directly into the peritoneal cavity of a rat or rabbit and observing its degradation over a period of 2 days to 12 months.

Alternatively, the degradation was also assessed by prefabricating a sterile implant, made by a process like solution casting, then surgically implanting the implant within an animal body. The degradation of the implant over time was monitored gravimetrically or by chemical analysis. The biocompatibility of the implant was assessed by standard histological techniques.

### EXAMPLES

The present invention will be further understood by reference to the following non-limiting examples. The various applications and processing shown here are exemplary only. Those skilled in the art will understand many other possible combinations which could be utilized for the purposes of the present invention.

The following non-limiting examples are intended to illustrate the properties of new biocompatible crosslinked polymers and their precursors, and their use in making several medical products. Those skilled in the art will appreciate that modifications can be made to these examples, drawings, illustrations and claims that are intended to fall within the scope the present invention.

### Example 1

In Vitro Degradation

Component A was PEG-Diacrylates (8,000 Da) and Component B was partially (˜85%) hydrolyzed PVA (30,000 Da) attached with hydroxyethyl methacrylate (HEMA)-lactate. The HEMA-glycolate-COOH crosslinker to PVA were prepared according to the reference Furch, M. et al., Polymer, 39(10):1977-1982 (1998).

First, 0.1 ml of Component A solution was mixed with (Component A solution contained 10% PEG-Diacrylates, 0.3% hydrogen peroxide, and 0.3% NVMA (N-vinyl N-methyl acetamide)) 0.2 ml of Component B solution in discs (Component B solution contained 15% PVA with HEMA-glycolate-COOH crosslinkers, 20 mg/ml Ferrous Ammonium Sulfate hexahydrate (Aldrich), 3% fructose, and 0.3% NVMA). Cure was instantaneous, and no discoloration of the gel occurred. The bond held during overnight soaking in distilled water.

Discs were incubated in phosphate-buffered saline, pH 7.4, at 37.degree. and 57.degree. C. At 57degree. C., half of the mass was lost at about 160 hrs, while at 37.degree. C., half the mass was lost at about 54 days. Mass loss was determined by rinsing the specimen, drying to constant weight, and correcting for the amount of buffer and salt present.

### Example 2

Sprayed Redox System

Using the above solutions, and with prepolymer concentrations varying from 5% to 10% in Component A solution and 10% to 30% in Component B solution, solution A was sprayed on semivertical surfaces, followed by solution B. Surfaces were petri dishes. The spraying procedure caused some foaming, but gels were formed on all surfaces. Because of running of the solutions down the surfaces, gels were thicker at the bottom but present throughout.

### Example 3

Comparison of Peroxygen Compounds

Reductant solutions contained 10% PEG-Diacrylates monomer and 8% by volume of a ferrous lactate solution, which itself contained 1% ferrous lactate and 12% fructose by weight in water. Oxidant solutions contained 15% PVA with HEMA-glycolate-COOH crosslinkers monomer and a constant molar ratio of oxidizer, which was, per ml of macromer solution, 10 microliters 30% hydrogen peroxide; 8.8 microliters tert-butyl peroxide; 15.2 microliters cumene peroxide; or 0.02 g potassium persulfate. 0.5 ml of reductant was mixed with 0.25 ml oxidizer, and time to gelation was noted. With hydrogen peroxide, gelling was nearly instantaneous, while with the others there was a short delay—about 1 second—before gelation. Doubling the t-butyl peroxide concentration also produced nearly instantaneous gelling. Hydrogen peroxide produced more bubbles in the gel than the others; persulfate had almost no bubbles. The bubbles in hydrogen peroxide may come directly from the reactant, as the other compounds have different detailed mechanisms of radical formation.

### Example 4

Effect of Reducing Sugars

Using the procedures of Example 3, the concentration of ferrous ion was reduced to 50 ppm, and the fructose was omitted. At 100 ppm HOOH in the oxidizing solution, gel time was increased to 3 to 4 seconds, with both Fe-gluconate and Fe-lactate, but gels were yellow. Addition of 125 ppm ascorbic acid to the reducing solution prevented the formation of the yellow color.

### Example 5

Coating of a Medical Device

A length of polyurethane tubing extrusion used for catheter shafts was dipped into an aqueous solution shown in example 1. The adherence was strong enough to survive sectioning of the tubing with a razor blade; photomicrography showed complete adherence of the gel to the tubing.

1. A mixed composition for forming a biocompatible and biodegradable hydrogel comprising two components;
wherein the first component comprises a core water soluble backbone having at least one hydroxyl group substituted with a pendant chain bearing a crosslinking group;
wherein the second component comprises a core water soluble region flanked by crosslinkers;
wherein the second component can be the crosslinkers alone.

2. The crosslinkers of the second component of claim 1 comprise biodegradable regions end caped with crosslinking groups;
wherein the crosslinking groups can crosslink with the crosslinking group of the first component, and with a crosslinking group on the same or a different first component; and
wherein the hydrogel formed from crosslinking of the first and second components degrades in vivo.

3. The end cap of the crosslinkers of clam 2 comprises one or more functional groups capable of cross-linking the macromers in vivo and in vitro.

4. The composition of claim 1 wherein the first and second components crosslink to form a hydrogel that fully degrades in vivo.

5. The composition of claim 1 wherein the first and second components crosslink to form a hydrogel that partially degrades in vivo.

6. The composition of claim 1 wherein at least one hydroxyl group of the core water soluble backbone is substituted with a modifier.

7. The core water soluble backbones or region of claim 1 preferably are poly(vinyl alcohol) and/or poly(ethylene glycol).

8. The core water soluble backbones or region of claim 1 can also be a co-polymer of PVA-PEG.

9. The composition of claim 1 wherein crosslinking of one or more of crosslinking groups can be initiated by a mechanism selected from the group consisting of thermal initiation, redox initiation, photoinitiation, or a combination thereof.

10. The composition of claim 6, wherein the modifier is selected from the group consisting of modifiers to change the hydrophobicity of the hydrogel, active agents and groups to allow attachment of an active agent, photoinitiators, modifiers to alter adhesiveness of the hydrogel, modifiers to impart thermo responsiveness to the hydrogel, and additional crosslinking groups.

11. The applications of the hydrogels formed of claim 1 can be used in the medical applications such as prevention of post-operative adhesions, surgical sealants, embolic therapies, controlled delivery of drugs, coating of medical devices such as vascular grafts, wound dressings and other medical applications.
